(19) United States
(12) Patent Application Publication
(10) Pub. No.: US 2025/0198960 A1
(43) Pub. Date: Jun. 19, 2025

(54) USE OF SENSORS BASED ON FUNCTIONALIZED CARBON NANOTUBES AND PROCESS FOR THE DETECTION OF CO2 IN GASEOUS ENVIRONMENTS

(71) Applicants: PETRÓLEO BRASILEIRO S.A. – PETROBRAS, Rio de Janeiro (BR); UNIVERSIDADE FEDERAL DE MINAS GERAIS - UFMG, Belo Horizonte (BR)

(72) Inventors: Natália Pereira REZENDE, Belo Horizonte (BR); Vinícius ORNELAS DA SILVA, Belo Horizonte (BR); Tiago Campolina BARBOSA, Belo Horizonte (BR); Cíntia Lima PEREIRA, Belo Horizonte (BR); Rodrigo Gribel LACERDA, Belo Horizonte (BR); Leonardo Cristiano CAMPOS, Belo Horizonte (BR); Myriano Henriques De OLIVEIRA JUNIOR, Belo Horizonte (BR); Thiago Henrique Rodrigues DA CUNHA, Belo Horizonte (BR); Cláudio Laudares Passos SILVA, Belo Horizonte (MG); Raissa Guerra RESENDE, Belo Horizonte (BR); Fabio Da Silva DUTRA, Rio de Janeiro (BR); Luiz Augusto De ANDRADE, Rio de Janeiro (BR)

(21) Appl. No.: 18/973,028

(22) Filed: Dec. 8, 2024

(30) Foreign Application Priority Data
Dec. 15, 2023 (BR) 1020230264832

(51) Int. Cl. G01N 27/12; G01N 1/24

(52) U.S. Cl. CPC G01N 27/128; G01N 1/24

(57) ABSTRACT

The present technology relates to the use of sensors based on functionalized carbon nanotubes for the detection of CO₂ in gaseous environments. Nanotubes have multiple layers and are functionalized with —OH and —COOH groups. The process to determine the concentration of CO₂ measures the electric current that passes through the sensor in the presence of different gases, the data obtained is treated and the concentration of CO₂ is obtained by methods involving artificial intelligence. The technology simultaneously has the following advantages: it identifies CO₂ in natural gas, uses short sensor exposure time ranges, allows similar results to be obtained even with small variations in the device due to the production process, uses optimized parameters and models to find the concentration of CO₂, the sensor can be miniaturized, is easily produced on large scale, is versatile, can be adapted to different substrates or analysis conditions, provides real-time results and is durable, maintaining accuracy even after several cycles in a row or after a long time of use. The technology can be used for identification of CO₂ in heterogeneous gaseous environments, such as natural gas.

### FIELD OF THE DISCLOSURE

The present technology relates to the use of sensors based on functionalized carbon nanotubes for the detection of CO₂ in gaseous environments. Nanotubes have multiple layers and are functionalized with —OH and —COOH groups. The process to determine the concentration of CO₂ measures the electric current that passes through the sensor in the presence of different gases, the data obtained is treated and the concentration of CO₂ is obtained by methods involving artificial intelligence. The technology simultaneously has the following advantages: it identifies CO₂ in natural gas, uses short sensor exposure time ranges, allows similar results to be obtained even with small variations in the device due to the production process, uses optimized parameters and models to find the concentration of CO₂, the sensor can be miniaturized, is easily produced on large scale, is versatile, can be adapted to different substrates or analysis conditions, provides real-time results and is durable, maintaining accuracy even after several cycles in a row or after a long time of use. The technology can be used for identification of CO₂ in heterogeneous gaseous environments, such as natural gas.

### BACKGROUND OF THE DISCLOSURE

A challenge in the field of exploration of oil or natural gas wells is the high content of contaminants, such as CO₂ present in the wells. CO₂ is the main cause of the greenhouse effect and corrosion of installations. Thus, it is essential to know its concentration for the formulation of solutions in its management, as large concentrations of CO₂ cannot be discharged directly into the atmosphere. It is important to monitor pipeline leaks or have greater control over the causes of corrosion in installations (J. J. M. OLIVEIRA. O PROBLEMA DA CORROSÃO POR CO₂ NOS TUBOS DE PRODUÇÃO DE POÇOS LOCALIZADOS NA PROVÍNCIA DO PRÉ-SAL. Monograph presented to the Postgraduate Course in Specialization in Oil and Natural Gas Engineering, at Faculdade do Centro Leste, 2015. https://www.researchgate.net/publication/308765198_O_Problema_da_Corrosao_por_CO2_nos_Tubos_de_Producao_de_Pocos_Localizados_na_Provincia_do_Pre-Sal).

In general, to identify the concentration of CO₂ and other components present in natural gas, some techniques are used, such as gas chromatography, electrochemical sensors, and semiconductor oxide or infrared sensors. Due to the high cost, short useful life, need for qualified labor, difficulty in withstanding the chemical and physical conditions of a heterogeneous gaseous environment, such as natural gas, and difficult integration into the production chain, these techniques do not fully meet the required demand. Thus, there is a need for CO₂ sensing using devices that are selective, small, portable, easy to integrate into the production and distribution chain, and can perform measurements in situ and in real time. In addition to the device, suitable methods are necessary for its use, including short and appropriate time ranges for measurement, calculations that are not affected by small differences due to large-scale production, the use of optimized parameters, and models capable of determining the concentration of CO₂ (Vinícius Ornelas da Silva). DESENVOLVIMENTO DE DISPOSITIVOS A BASE DE NANOMATERIAIS DE CARBONO PARA SENSORIAMENTO DE CO₂. Dissertation presented to the Postgraduate Program in Physics, Federal University of Minas Gerais, 2023).

Carbon nanotubes are allotropes of carbon with a cylindrical nanostructure. They can have one layer or multiple concentric layers. Their functionalization happens when groups of atoms bind together on its surface, allowing the carbon nanotube to better interact with the medium in which it is inserted. Carbon nanotubes have enormous potential in sensor applications due to their excellent mechanical and electrical properties, large surface area and resistance to corrosive environments. Such characteristics make these materials natural candidates for a novel nanosensor platform. The properties of nanotubes depend on their structure or chirality, and may have, e.g., metallic or semiconductor characteristics. When there are several nanotubes of different chiralities deposited in a medium forming a nanotube film, this film can exhibit a metallic behavior. When gases come into contact with the nanotube film, their transport properties will change, and it is possible to associate this change with the nature of the gas.

### STATE OF THE ART

In this sense, there are some documents in the state of the art that disclose devices and methods aimed at identifying molecules in gaseous environments.

The patent document US2023121903A1, with priority date Oct. 17, 2022, entitled “Device and analysis method for appreciating and identifying smells”, describes a device comprising an airflow system, a chamber with an array of sensors of molecules present in the air, a configurable sensor interface, a microcontroller, a memory, and a metal mesh filter. The device collects data from molecules present in the air, processes, stores and transfers the data to the user in real time. The data collected by the device is linked to a methodology involving artificial intelligence to identify the aroma of the air sample. However, the device described in the document US2023121903A1 is designed to work in the presence of atmospheric air, not demonstrating to withstand corrosive gaseous environments, and may not maintain its accuracy after several consecutive cycles or after a long time of use in corrosive environments with natural gas, uses an array of sensors to distinguish several molecules, increasing the complexity of the device or data processing method, uses air as a desorption gas for sensor molecules, and the air may have impurities that react with the sensor, reducing its lifespan or its accuracy in identifying some gases, uses non-optimized input parameters in the artificial intelligence model for the detection of CO₂, such as the area or the maximum value of the normalized gain or electrical current curve passing through the sensor, which reduces the accuracy in identifying CO₂ concentration or does not favor a time range that contributes to the response speed of the sensor array, and the device and the process are not optimized for the detection of gases like CO₂ in the presence of heterogeneous gases, such as natural gas.

The patent document EP2912454A1, with priority date Oct. 29, 2012, entitled “Functionalized nanotube sensors and related methods”, describes a sensor and method based on nanotubes with metal oxides for identification of volatile organic compounds and biomarkers in a fluid environment. The sensor has a power source configured to apply an electrical voltage to the nanotube array and a current sensor configured to monitor and detect changes in a response current that varies after binding with the composite target. However, the technology discussed in the document EP2912454A1 focuses on the identification of molecules that can be used as biomarkers, not presenting properties that withstand corrosive gaseous environments, and may not maintain precision even after several consecutive cycles or after a long time of use, it requires an array of sensors to distinguish several molecules, increasing the complexity of the device or data processing method, uses input parameters not optimized for CO₂ detection in the artificial intelligence model, such as maximum gain area or value or normalized electric current, decreasing the precision in identifying the concentration of CO₂ or not favoring a time range that contributes to the actuation speed of the sensor array, uses non-normalized measured parameters, making the results obtained vary depending on the method of large-scale sensor production or small variations in its use, in addition to the device in the process not being optimized for the detection of gases such as CO₂ in the presence of heterogeneous gases, such as natural gas.

The use of sensors based on functionalized carbon nanotubes for the detection of CO₂ in gaseous environments was not found in the state of the art. The present disclosure relates to the use of sensors based on multilayer carbon nanotubes functionalized with —OH and —COOH groups for the detection of CO₂ in gaseous environments. The technology uses methods, such as data processing and artificial intelligence, that optimize the identification of the concentration of CO₂ in heterogeneous environments such as natural gas, presenting the following advantages together: identification of CO₂ in heterogeneous gases, such as natural gas, use of optimized input parameters in the artificial intelligence model for the detection of CO₂, such as the area or the maximum value of the normalized gain or electric current curve passing through the sensor, increase of the precision in identifying CO₂, allows the use of short sensor exposure time ranges, allowing the obtainment of similar results even with small variations of the device due to the production process or its handling, is fast, can be miniaturized, is easy to produce on a large scale, is versatile, can adapt to different substrates or analysis conditions, and is durable, maintaining accuracy even after several consecutive cycles or after a long time of use.

### BRIEF DESCRIPTION OF THE FIGURES

FIG. 1 shows a system for detection of CO₂ in gaseous environments comprising a sensor (0), a film (1) of multilayer carbon nanotubes functionalized with —OH and —COOH groups, an insulating substrate (2), metal contacts (3), a gas passage chamber (4), an insulation surface of the internal environment (5), an inlet for gases (6), an outlet for gases (7), openings for the passage of electrical cables (8), a computer (9), a compartment for different gases (10), control valves (11), vacuum pump (12), ammeter (A) and a voltage source (V) according to an embodiment of the disclosure.

FIG. 2 shows a representation of the sensor (0). FIG. 2(a) shows sensor (0) with a film (1) of multilayer carbon nanotubes functionalized with —OH and —COOH groups, an insulating substrate (2), metal contacts (3), ammeter (A) and a voltage source (V). FIG. 2(b) shows an ammeter (A), a voltage source (V) and a representation of the carbon nanotubes (NTC) of the film (1) forming paths in which an electric current passes between the electrical contacts (3) according to an embodiment of the disclosure.

FIG. 3 shows images of carbon nanotubes according to an embodiment of the disclosure. The scale of FIG. 3(a) is e3a=1 μm. The scale of FIG. 3(b) is e3b=20 nm.

FIG. 4 shows a graph of current I(t), given in Amperes (A), as a function of time t, given in seconds(s). The interval between 180 s and 240 s, marked in gray, demarcates the instant where the gas flow with a concentration of 10% CO₂ was turned on according to an embodiment of the disclosure.

FIG. 5 shows graphs of the gain G(t) as a function of time t, given in seconds(s). The temperature T is 30° C. and the concentration of CO₂ is 10%. In FIG. 5(a), the black arrow represents the moment (ON) at which the gas flow with CO₂ was turned on according to an embodiment of the disclosure, and the colored arrows represent the moment (OFF) at which the gas flow with CO₂ was turned off. The different colors of each curve represent the following time ranges in which the sensor (0) came into contact with the gas flow containing CO₂: 30 s, 60 s, 90 s, 150 s and 300 s. FIG. 5(b) shows a gain graph G(t) as a function of time t, where sensor (0) is exposed to the gas flow containing CO₂ for 60 s. FIG. 5(c) shows a gain graph G(t) as a function of time t, where sensor (0) is exposed to the gas flow containing CO₂ for 300 s. In FIGS. 5(b) and 5(c), the different colors of each curve represent the following concentrations of CO₂: 0%, 5%, 10% and 15%.

FIG. 6 shows the gain graph G(t) as a function of time t, given in seconds(s) according to an embodiment of the disclosure according to an embodiment of the disclosure. Each color of the curve represents the percentage of CO₂ in the natural gas measured every 10%, ranging from 0% to 100%.

FIG. 7 shows, in a non-limiting manner, the gain graph G(t) as a function of time t, given in seconds(s). The data in FIGS. 7(a)-7(h) were measured in December 2021, January 2022, February 2022, March 2022, April 2022, May 2022, June 2022, and July 2022, respectively. The colors on each curve of the graphs represent the concentrations 0%, 5%, 78, 10%, and 12% of CO₂ in the natural gas.

FIG. 8 shows graphs that demonstrate the reproducibility of the sensor (0). FIGS. 7(a)-7(d) show the gain G(t) as a function of time t, given in seconds(s) according to an embodiment of the disclosure. Tin1 and tin2 represent two inks obtained with the same methodology. Rep1, Rep2, and Rep3 represent different sensors (0) reproduced for testing. The colors on each curve of the graphs represent the concentrations 0%, 5%, 7%, 10%, and 12% of CO₂ in the natural gas. FIG. 8(e) shows a graph of the average maximum gain GmaxMed as a function of the concentration Con of CO₂ in natural gas, and FIG. 8(f) shows a graph of the average gain area AreMed as a function of the concentration Con of CO₂ in the natural gas.

FIG. 9 shows graphs that demonstrate the effects of sensor (0) non-saturation according to an embodiment of the disclosure. FIG. 9(a) shows the gain G(t) as a function of time t, given in seconds(s). FIG. 9(b) shows the maximum mean value of the gain GmaxMed as a function of the concentration Con of CO₂ in the natural gas. FIG. 9(c) shows the value of the average gain area AreMed as a function of the concentration Con of CO₂ in the natural gas. The colors on each curve of the graphs represent the concentrations 0%, 5%, 7%, 10%, 12% and 15% of CO₂ in the natural gas.

### DETAILED DESCRIPTION OF THE TECHNOLOGY

The present technology relates to the use of sensors based on functionalized carbon nanotubes for the detection of CO₂ in gaseous environments. Nanotubes have multiple layers and are functionalized with —OH and —COOH groups. The process to determine the concentration of CO₂ measures the electric current that passes through the sensor in the presence of different gases, the data obtained is treated and the concentration of CO₂ is obtained by methods involving artificial intelligence. The technology simultaneously has the following advantages: it identifies CO₂ in natural gas, uses short sensor exposure time ranges, allows similar results to be obtained even with small variations in the device due to the production process, uses optimized parameters and models to find the concentration of CO₂, the sensor can be miniaturized, is easily produced on large scale, is versatile, can be adapted to different substrates or analysis conditions, provides real-time results and is durable, maintaining accuracy even after several cycles in a row or after a long time of use. The technology can be used for identification of CO₂ in heterogeneous gaseous environments, such as natural gas.

Sensors based on functionalized carbon nanotubes can be used for the detection of CO₂ in gaseous environments. The process for detecting CO₂ using the sensors of this technology can be carried out through the following steps:
a) Connect the sensor (0) to a gas passage chamber (4), where the sensor (0) comprises a film (1) of multilayer carbon nanotubes functionalized with —OH and —COOH groups, deposited on an insulating substrate (2) and with electrically connected ends in metal contacts (3), the gas passage chamber (4) comprising an insulating surface of the internal environment (5), one inlet for gases (6) and one outlet for gases (7);
b) Apply an electric potential difference to the metal contacts (3) of the sensor (0) using a voltage source (V);
c) Record in a computer (9) the current I_{base}(t) as a function of the time t that passes through the sensor (0), measured through the ammeter (A);
d) Expose the sensor (0) to an inert gas or low-pressure environment within the gas passage chamber (4) until the current I_{base}(t) passing through the sensor (0) tends to have a constant behavior over time t;
e) Expose the sensor (0) to an atmosphere with a gas flow containing CO₂, controlling the valves (11) of the different gas compartments (10), and calculate the current I_{CO2}(t) that passes through the sensor (0);
f) Turn off the gas flow containing CO₂ and calculate the gain G(t) according to equation eq1, where the minimum exposure time tₑₓₚ of the sensor (0) to the gas flow containing CO₂, preferably between tₑₓₚ=60 s and tₑₓₚ=300 s, is chosen to favor the distinction of G(t) for each value of the concentration of CO₂;

$$G ( t ) = \vert ( {I}_{C O 2} ( t ) - {I}_{base} ( t ) ) \vert / {I}_{base} ( t ) ; \qquad eq1$$

g) Plot a gain graph G(t) as a function of time t;
h) Obtain the area A_{G} under the curve of the gain graph G(t) and the maximum gain value Gₘₐₓ from the gain value G(t) obtained in steps “f” and “g”;
i) Submit the features area A_{G} and maximum gain value Gₘₐₓ obtained in step “h”, as input data for the trained model to obtain concentrations, where the label of each result assigned by the trained model for obtaining concentrations is the predicted concentration of CO₂.

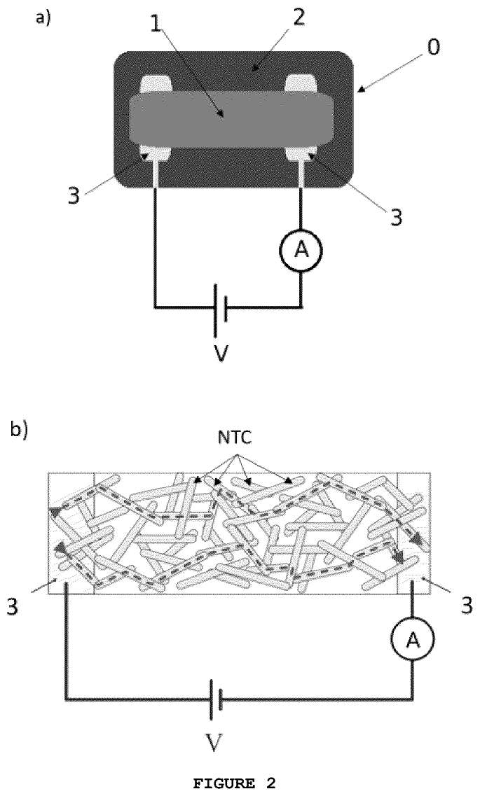

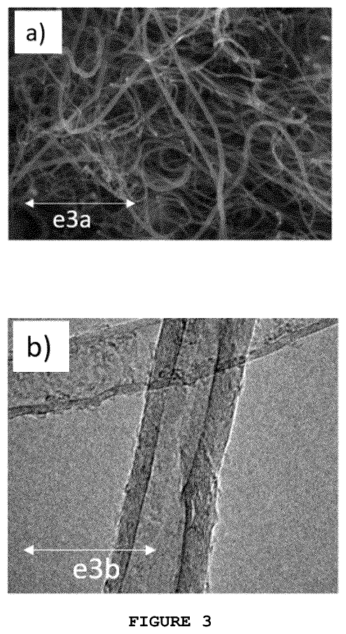

In step (a), the film (1), present in sensor (0), has functionalized carbon nanotubes with —OH and —COOH groups, where all the nanotubes are leaning against and entangled with each other, ensuring that there is always a pathway for electric current to flow between the metal contacts (3), as described in FIG. 2(b) and FIG. 3. Metal contacts can be constructed of materials such as gold, copper, silver, or platinum. The insulating substrate (2) accommodates the film (1) in addition to protecting it electrically, and can be constructed of various insulating materials such as silicon oxide, glass, papers, polymers, resins or phenolite. In the presence of gases containing CO₂, CO₂ interacts with carbon nanotubes, especially with the —OH and —COOH groups, changing their electrical properties, enabling the detection of CO₂. The gas passage chamber (4) isolates, thermally and chemically, the internal gaseous environment through the insulation surface of the internal environment (5). The inlet for gases (6) allows a desired gas to enter the chamber for gas passage (4) and the outlet for gases (7) allows gases to be expelled, and control the pressure difference in the chamber. The vacuum pump (12) assists in the removal of gases from the gas passage chamber (4). The openings for the passage of electrical cables (8) allow the passage electrical connections without gas leakage, enabling the operation of the sensor (0) in the gas of interest.

In steps (b) and (c), a voltage source V allows the passage of current I_{base}(t) on the sensor (0), which is measured by the ammeter (A) and recorded in the computer (9). The change in current I_{base}(t) recorded in the computer (9) will indicate that different gases have passed through the sensor.

In step (d), exposing the sensor (0) to an inert gas or low pressure environment within the gas passage chamber (4) removes unwanted impurities that can interact with the functionalized carbon nanotubes and impair the sensor measurement (0). The value of the current I_{base}(t) becoming constant over time t is an indication that the sensor (0) is ready to be used to identify other gases. The flow of inert gas or the generation of low pressure can be maintained if new measurements with the sensor (0) are necessary. In this case, the control of the density of the gas containing CO₂ is favored with the injection of inert gases or the generation of low pressure inside the gas passage chamber (4).

In step (e), the sensor (0) is subjected to a gas containing CO₂, such as natural gas, and the current I_{CO2}(t) passing through the sensor (0) is recorded. Current I_{CO2}(t) will tend to decrease over time in the presence of CO₂, demonstrating that the gas is interacting with the carbon nanotubes.

The current passing through the sensor (0) may fluctuate according to its production and deterioration over time, requiring the use of the gain G(t) described in step (f), favoring the method of identification of CO₂ in heterogeneous gases. The minimum time tₑₓₚ of exposure of the sensor (0) to the gas flow containing CO₂, preferably between tₑₓₚ=60 s and tₑₓₚ=300 s, is chosen in a way that favors the distinction of G(t) for each value of the concentration of CO₂ contributing to the increase of the speed in the identification of gases.

The graph of G(t) as a function of time t, plotted in step (g), is used to calculate parameters that facilitate data analysis, such as the area A_{G} under the gain graph G(t) and the maximum gain value Gₘₐₓ, as shown in step (h). With the area A_{G} under the gain graph G(t) and the maximum gain value Gₘₐₓ, it is possible to obtain the concentration of CO₂ through trained models designed to determine concentrations of CO₂.

In step “b”, the difference in electrical potential through a voltage source (V) is between 1 mV and 100 V.

The segment of the gain curve G(t) with the greatest relevance to obtain the concentration of CO₂ can be obtained by considering only the values of the gain G(t) at the moments when the sensor (0) comes into contact with the gas flow containing CO₂. This procedure favors the obtainment of concentrations of CO₂ providing greater precision in the identification of the concentration of CO₂ by using different methods, such as artificial intelligence.

The model trained to obtain concentrations is based on the features area A_{G} under the gain G(t) and the maximum gain value Gₘₐₓ where each predicted class is labeled with the concentration of CO₂ and can be selected from the following:
1. a decision tree classification model;
2. a Random Forest trained classification model;
3. a linear regression model.

After the concentration of CO₂ has been detected, the sensor (0) can be recovered by desorption of unwanted elements from the sensor (0) by following the steps below:
I. Connect the sensor (0) to a gas passage chamber (4), where the sensor (0) comprises a film (1) of multilayer carbon nanotubes functionalized with —OH and —COOH groups, deposited on an insulating substrate (2) and with electrically connected ends in metal contacts (3), the gas passage chamber (4) comprising an insulating surface of the internal environment (5), one inlet for gases (6) and one outlet for gases (7);
II. Apply an electric potential difference to the metal contacts (3) of the sensor (0) using a voltage source (V);
III. Record in a computer (9) the current Ides (t) as a function of the time t that passes through the sensor (0), measured through the ammeter (A);
IV. Expose the sensor (0) to an inert gas or vacuum environment within the gas passage chamber (4) until the current I_{base}(t) passing through the sensor (0) tends to have a constant behavior over time t.

In steps (I), (II) and (III), the sensor (0) is placed in the chamber (4), a voltage is applied, and the current is recorded. In step (IV), impurities that are trapped among the functionalized carbon nanotubes can be removed. When the current value Ides (t) is constant over time, the sensor is recovered and ready for new use.

Exposure of the sensor (0) to an atmosphere with inert gas flow, such as argon, nitrogen, or helium, occurs by allowing inert gas to exit a compartment for different gases (10) through control valves (11), enter the gas passage chamber (4) through the inlet for gases (6), and exit through an outlet for gases (7).

The formation of a vacuum or a low-pressure environment occurs by reducing the gas inflow at the inlet for gases (6) and allowing gas to exit through the outlet for gases (7) with the aid of a vacuum pump (12).

The present technology can be better understood through the following not limiting examples.

### Example 1—Fabrication of the Sensor

Multi-walled carbon nanotubes are produced by the chemical vapor phase deposition (CVD) technique. To perform the synthesis of carbon nanotubes by CVD growth, there is a system consisting of a hot-wall furnace with a quartz tube in its interior. This tube rotates around its axis of rotation at a temperature of approximately 730° C. A constant flow of argon (2 l/min) creates an inert atmosphere under atmospheric pressure, and carries impurities generated by the growth process inside the tube to the exhaust. A constant flow of ethylene gas (1.5 l/min) is also used as a carbon source for the synthesis of carbon nanotubes. The system is continuously fed with a catalyst consisting of Fe and Co oxide nanoparticles, supported by particles of Al₂O₃, through a feeder connected to the tube inlet. The quartz tube is kept inclined 10° to horizontal and rotates at a speed of 6 RPM to promote the transport of the catalyst through the furnace hot zone. The carbon nanotubes are formed in the catalyst and transported for storage in a container attached to the end of the tube. After the synthesis, the NTC are functionalized. During the functionalization, —OH and —COOH groups are added to the walls of carbon nanotubes through an acid (HNO₃/H₂SO₄) treatment process, which is performed in an ultrasound bath under controlled temperature. The functionalization process is necessary to dilute the carbon nanotube in water or organic solvents. After that, the material is washed and centrifuged several times, and then dried in a kiln (100° C. for 24 hours) and ground in an automated pistil mill.

The carbon nanotubes are dispersed in deionized water at an initial concentration of 2% w/v, in an ultrasound bath for 4 hours. The obtained suspension is centrifuged at 300 RPM for 30 minutes, and the supernatant material is separated from the precipitate. Then, the concentration of the supernatant is measured. In general, after this process, the ink has a concentration of around 1.2-1.3% w/v. Finally, the ink is diluted in isopropyl alcohol until achieving the standard concentration of 0.6% w/v.

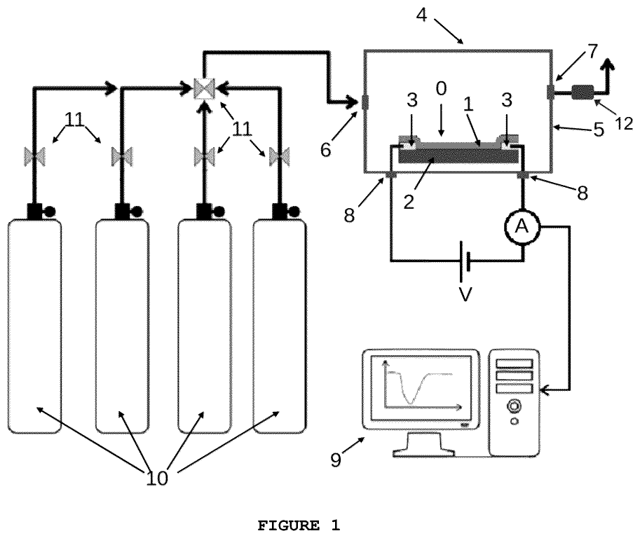

Once the process of making the standard nanotube ink is finished, the process of producing the sensors begins. To achieve this, a mask was cut using a laser printer based on a design created on a computer, for the evaporation of the electrical contacts (length=1 mm and width=3 mm). Another mask was made to subsequently paint the conductive channel (length of the carbon nanotube channel=2 mm and width of the carbon nanotube channel=1 mm). Using the thermal evaporation method, electrical contacts (metal contacts (3) from FIGS. 1 and 2) of 3 nm chromium (Cr) and 50 nm gold (Au) are formed on the surface of a substrate composed of a layer of silicon oxide on a silicon substrate (SiO₂/Si), represented by the insulating substrate (2) in FIGS. 1 and 2. Upon completing the deposition of the metal contacts (3), the conductive channel of the device is painted using nanotube ink through an airbrush integrated with axes that move in the cartesian XY plane, controlled by a computer. The substrate is located just below the airbrush, on a thermal base at 140° C., so that solvents evaporate and prevent the nanotubes from diffusing across the surface and agglomerating. Thus, it is possible to perform the deposition of nanotube films (1) in a precise and controlled manner. One of the forms of reproducibility control adopted is the measurement of the resistance of the conductive channel during the painting process, where the resistance will be proportional to the height of the deposited layers, since the length and width are fixed, defined by the mask size. Typically, sensors (0) are painted with three coats of paint, with resistance in the range of 1-2 kΩ. The sensor (0) connected to the voltage source V and the ammeter A can be seen in FIGS. 1 and 2.

After painting, the substrate is inserted into a chip, where the contact between the conductive channel and the pins that make up the chip is established, constructing the sensor (0). Once the sensor (0) is completed, it is inserted into the measurement system. FIG. 3 shows scanning transmission electron microscopy images. FIG. 3(a) shows the morphology of the multi-walled nanotubes of a painted device. The nanotubes have a length and entanglement such that throughout the entire ink, there are always pathways for electric currents to flow through the sensor (0). FIG. 3(b) shows a concentric multilayer carbon nanotube in a transverse position to another multilayer carbon nanotube. With this production technique, sensors (0) can be manufactured on a large scale.

### Example 2—Detection of CO₂ in Natural Gas

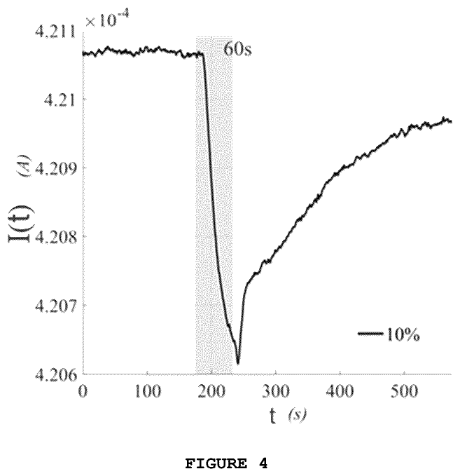

As an example of heterogeneous gas, natural gas was used. The difference in gas flows during the measurement generates a current pattern I(t) as a function of time t similar to the one shown in FIG. 4. In FIG. 4, until time t=180 s, the sensor (0) is exposed to the flow of argon gas, an example of inert gas, and has an applied voltage of 1 V between the terminals (3), generating a current I_{base}(t) tending to have a constant pattern. In the time range from t=180 s to t=240 s, the argon gas is turned off and the natural gas is turned on, and it is possible to see the drop in the value of the current I_{CO2}(t). From time t=240 s on, natural gas is turned off and argon gas is turned on, causing the current to increase until the current becomes constant in time.

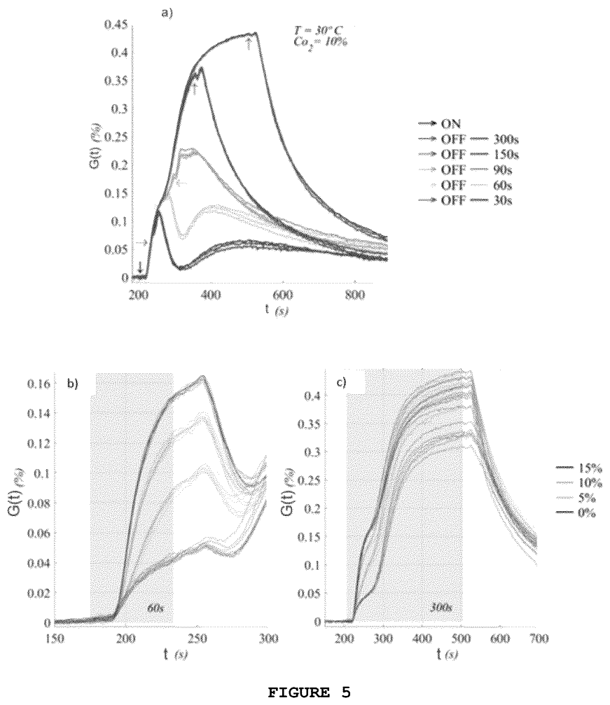

With the value of the currents I_{base}(t) and I_{CO2}(t), the gain G(t)=|(I_{CO2}(t)−I_{base}(t))|/I_{base}(t) is calculated. FIG. 5 shows the gain G(t) for various time ranges in which natural gas is on, at a fixed temperature of 30° C. and atmospheric pressure. FIG. 5(a) shows the gain G(t) for various exposure times while maintaining the concentration of CO₂ of 10% in natural gas. The fast state condition (60 s) can be seen in FIG. 5(a) between the black arrow (when the natural gas flow is turned on) and yellow (when the natural gas flow is turned off). The slow regime condition (300 s) is between the black and purple arrow. FIG. 5(b) shows the gain G(t) with the exposure time t=60 s of the sensor (0) to natural gas for various concentrations of CO₂. FIG. 5(c) shows the gain G(t) with the exposure time t=300 s of the sensor (0) to natural gas for various concentrations of CO₂. The exposure time value between tₑₓₚ=60 s and tₑₓₚ=300 s is chosen in a way that favors the distinction of G(t) for each value of the concentration of CO₂. The fast regime where the exposure time is tₑₓₚ=60 s favors a good identification between concentrations in a short measurement time. The value of the gain G(t) analyzed will be only in the time range of interest in which the sensor (0) had contact with CO₂.

With gain G(t), the features area of the gain A_{G} and the maximum gain value Gₘₐₓ are obtained. For the concentration of 10% of CO₂ and the exposure time of the sensor (0) to natural gas of t=60 s, we have the area A_{G} 23 s and the maximum gain value Gₘₐₓ Of 0.135. The values A_{G} and Gₘₐₓ are submitted as input data to the trained decision tree and Random Forest models, as well as to the linear regression model, where each predicted class is labeled with the concentration of CO₂.

To train the decision tree and Random Forest models, the dataset is initially split into training and testing data using the cross-validation technique. Cross-validation is a resampling method that uses different pieces of data to test and train a model in different iterations. The training data is used to teach the model how to identify the outputs when using a new set of inputs. The test data is used to validate the system. The training data is divided into 5 parts. Initially, the first part is used to train and the others to validate the method, and a hit rate is calculated. Then, the second part is used to train and the others to validate the algorithm, and a new hit rate is calculated. This process is done repeatedly up to the fifth part of the training data, calibrating the parameters of the method. In the end, a mean of the hit rate of the processes carried out from the first to the fifth part is calculated, and the best parameter is determined where the least error is obtained. It is observed that the main characteristic of the cross-validation method is to always train and test the algorithm on different dataset configurations, i.e., it is the equivalent of testing and training the algorithm with new datasets in each process performed from the first part of the data to the fifth part; this is what makes the cross-validation method a reliable measure of the method performance. Then, once the best parameter for this particular method is selected, it is retrained, and finally evaluated with new test data being determined as the final model.

### Example 3—Sensor Recovery

When we perform multiple measurements, unwanted molecules can get stuck in the nanotubes present in the film (1), and it is necessary to recover the sensor (0) in order to make a new measurement. Sensor recovery (0) was performed by subjecting the sensor to 110° C. for 8 hours under argon atmosphere for moisture removal. It turns out that, after recovery, the resistance of the sensor (0) decreased from 2.6 kΩ to 2.4 kΩ. This may be associated with improved quality of electrical contacts, as well as desorption of contaminant molecules from the sensor surface (0).

### Example 4—Sensor Characterization

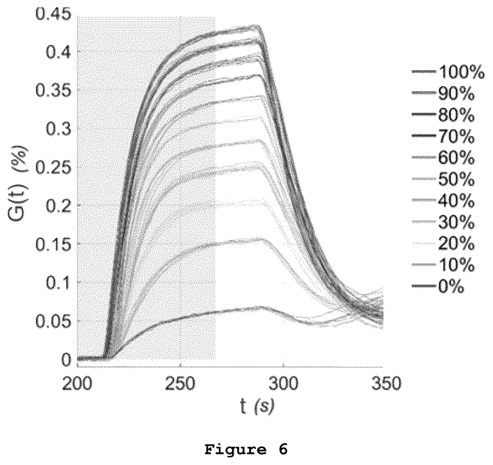

Operating range: in order to verify the operating range of the sensor, gain curves G(t) over time t were collected under a condition where the sensor was exposed to random concentrations ranging from 0% CO₂ (pure natural gas) to 100% CO₂ over a period of 60 seconds. FIG. 6 shows the sensor's ability to distinguish all applied concentrations, without saturation, even for high values such as 100% CO₂.

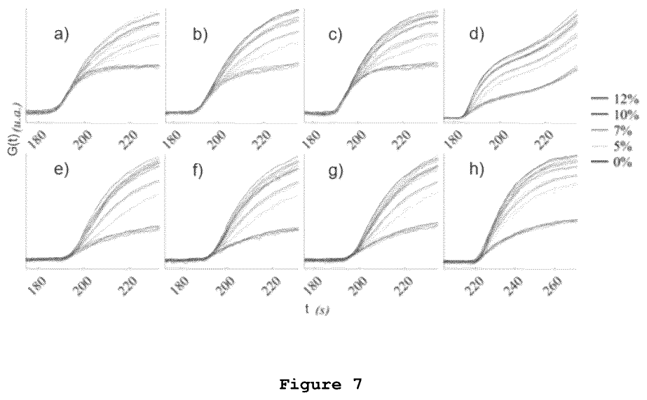

Lifespan: an important feature of sensors is the lifespan. Sensors that need constant replacement and maintenance may not be very interesting due to costs, repair downtime or handling by qualified labor. Thus, it is necessary to evaluate the sensor lifespan. To do this, different rounds of experiments were carried out that add up to more than 5,000 measurements on the same device over 8 months. FIG. 7 shows some gain curves G(t) over time t for each of the months of measurements. The lifespan of the sensors was measured during the following months: December 2021 (FIG. 7(a)), January 2022 (FIG. 7(b)), February 2022 (FIG. 7(c)), March 2022 (FIG. 7(d)), April 2022 (FIG. 7(e)), May 2022 (FIG. 7(f)), June 2022 (FIG. 7(g)) and July 2022 (FIG. 7(h)). It is worth mentioning that, throughout this period, the sensor was removed from the chamber and exposed to the air numerous times. It is observed that, despite variations in the curve profiles in some months, the sensor continues to distinguish concentrations such as 0%, 5%, 7%, 10%, and 12%, adopted for these tests, demonstrating the robustness of the developed sensor and its potential for long-term applications.

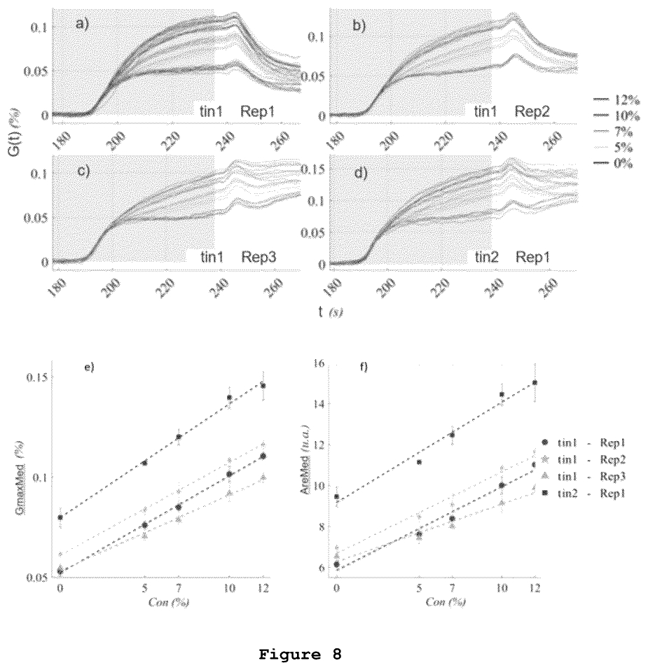

Reproducibility of the sensors: to test the reproducibility of the production of sensors, four different devices were manufactured. In each round of measurements for a given sample, a total of 30 curves were collected, with 5 measurements for each concentration, from 0% to 12%. As can be seen in FIG. 8, all samples exhibit a similar overall behavior, along with satisfactory separation between the different concentrations of CO₂ in natural gas. FIGS. 8(e) and 8(f) present the calibration curves in terms of the average maximum gain GmaxMed versus concentrations Con and average area AreMed versus concentration Con, respectively. The symbols in red, light green and cyan, correspond to the reproduction devices Rep1, Rep2 and Rep3 painted with the same ink, called ink 1 (tin1), and the symbol in purple corresponds to the reproduction device 4 painted with a new ink produced following the same preparation procedures, which was named ink 2 (tin2). All curves are linear, but not all of them have close slope values. This is not a problem, as each sensor has its calibration for operation. This demonstrates that different devices and different batches of paint manufacturing do not significantly interfere in the detection and identification of the concentrations of CO₂ in natural gas.

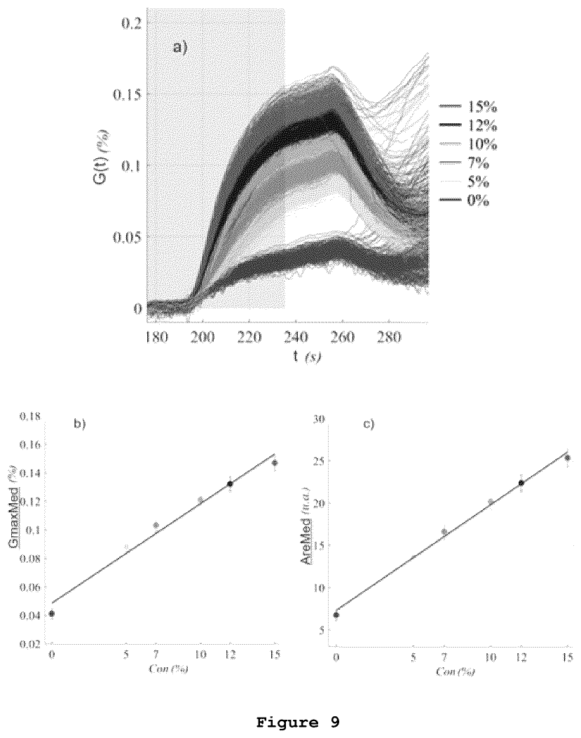

Saturation effect: sensors can present saturation conditions after repeated exposure to the working gas, directly interfering with the precision of the measurements. FIG. 9(a) shows 2,000 gain curves g (t) over time t for concentrations of 0, 5, 7, 10, 12 and 15% of CO₂ randomly applied over the entire 15-day period. It was observed that, over time, all curves undergo a global shift to lower gain values. However, the difference between maximum gains for adjacent concentrations remains basically constant, not interfering with the differentiation capacity of CO₂ contaminations. In addition, FIG. 9(b) and FIG. 9(c) present the parameters of mean maximum gain and mean area of the gain curve by concentration, as well as their standard deviation, calculated in the gas exposure interval, for the 2,000 curves collected. As it can be seen, the standard deviation bar in each concentration does not overlap the adjacent concentration, which is another indication of the ability to differentiate concentrations using these parameters. This result demonstrates that the sensor can work with large numbers of measurements without significant changes in the detection and identification of concentrations of CO₂ in natural gas.

In summary, the use of the sensor (0) has several properties such as a lifespan of more than eight months without losing efficiency, has good reproducibility, does not present a saturation over time that prevents its use in the identification of concentration of CO₂ and has a good operating range identifying from 0% to 100% of CO₂ in natural gas (Vinícius Ornelas da Silva. DESENVOLVIMENTO DE DISPOSITIVOS A BASE DE NANOMATERIAIS DE CARBONO PARA SENSORIAMENTO DE CO₂. Dissertation presented to the Postgraduate Program in Physics, Federal University of Minas Gerais, 2023).

1. Use of sensors based on multilayer carbon nanotubes functionalized with —OH and —COOH groups, comprising for detecting CO₂ in gaseous environments.

2. A process for the detection of co: in gaseous environments to use multilayer carbon nanotubes functionalized with —OH and —COOH groups, the process comprising the following steps:
a) Connect the sensor to a gas passage chamber, where the sensor comprises a film of multilayer carbon nanotubes functionalized with —OH and —COOH groups, deposited on an insulating substrate and with electrically connected ends in metal contacts, the gas passage chamber comprising an insulating surface of the internal environment, one inlet for gases and one outlet for gases;
b) Apply an electric potential difference to the metal contacts of the sensor using a voltage source;
c) Record in a computer the current I_{base}(t) as a function of the time t that passes through the sensor, measured through the ammeter;
d) Expose the sensor to an inert gas or low-pressure environment within the gas passage chamber until the current I_{base}(t) passing through the sensor tends to have a constant behavior over time t;
e) Expose the sensor to an atmosphere with a gas flow containing CO₂, controlling the valves of the different gas compartments, and calculate the current I_{CO2}(t) that passes through the sensor;
f) Turn off the gas flow containing CO₂ and calculate the gain G(t) according to equation eq1, where the minimum exposure time tₑₓₚ of the sensor to the gas flow containing CO₂, preferably between tₑₓₚ=60 s and tₑₓₚ=300 s, is chosen to favor the distinction of G(t) for each value of the concentration of CO₂;
$$G ( t ) = \vert ( {I}_{C O 2} ( t ) - {I}_{base} ( t ) ) \vert / {I}_{base} ( t ) ; \qquad eq1$$
g) Plot a gain graph G(t) as a function of time t;
h) Obtain the area A under the curve of the gain graph G(t) and the maximum gain value Gₘₐₓ from the gain value G(t) obtained in steps “f” and “g”;
i) Submit the features area A_{G} and maximum gain value Gₘₐₓ obtained in step “h”, as input data for the trained model to obtain concentrations, where the label of each result assigned by the trained model for obtaining concentrations is the predicted concentration of CO₂.

3. The process according to claim 2, wherein step “b”, the difference in electric potential is applied through a voltage source (V) from 1 mV to 100 V.

4. The process according to claim 2, wherein step “f,” obtaining the segment of the gain curve G(t) is more relevant for determining the concentration of CO₂ by considering only the values of the gain G(t) at the moments when sensor (0) comes into contact with the gas flow containing CO₂.

5. The process according to claim 2, wherein step “i”, the model trained to obtain concentrations is a trained decision tree classification model based on the features area A_{G} under the gain G(t) and the maximum gain value Gₘₐₓ in which each predicted class has as its label the concentration of CO₂.

6. The process according to claim 2, wherein step “i”, the model trained to obtain concentrations is a trained Random Forest classification model based on the features area A_{G} under the gain G(t) and the maximum gain value Gₘₐₓ in which each predicted class has as its label the concentration of CO₂.

7. The process according to claim 2, wherein step “i”, the model trained to obtain concentrations is a linear regression classification model based on the features area A_{G} under the gain G(t) and the maximum gain value Gₘₐₓ in which each predicted class has as its label the concentration of CO₂.

8. The process according to claim 2, wherein after step “i”, the sensor recovery is carried out through the desorption of unwanted elements of the sensor (0) following the steps below:
a) Connect the sensor to a gas passage chamber, where the sensor comprises a film of multilayer carbon nanotubes functionalized with —OH and —COOH groups, deposited on an insulating substrate and with electrically connected ends in metal contacts, and gas passage chamber comprising an insulating surface of the internal environment, one inlet for gases and one outlet for gases;
b) Apply an electric potential difference to the metal contacts of the sensor using a voltage source;
c) Record in a computer the current Ides (t) as a function of the time t that passes through the sensor, measured through the ammeter;
d) Expose the sensor to an inert gas or vacuum environment within the gas passage chamber until the current I_{base}(t) passing through the sensor tends to have a constant behavior over time t.

9. The process according to claim 2, wherein the exposure of the sensor (0) to an atmosphere with inert gas flow, such as argon, nitrogen, or helium, occurs by allowing inert gas to exit a compartment for different gases (10) through control valves (11), enter the gas passage chamber (4) through the inlet for gases (6), and exit through an outlet for gases (7).

10. The process according to claim 2, wherein the formation of a vacuum or a low-pressure environment occurs by decreasing the gas inflow at the inlet for gases and allowing gas to exit through the outlet for gases with the aid of a vacuum pump.
